(19) United States
(12) Patent Application Publication
(10) Pub. No.: US 2024/0156707 A1
(43) Pub. Date: May 16, 2024

(54) Hair growth essence and its preparation method

(71) Applicant: Shenzhen Okary Biotech Co.,Ltd., Shenzhen (CN)

(72) Inventor: ZEDA CHEN, Shenzhen (CN)

(21) Appl. No.: 17/985,927

(22) Filed: Nov. 14, 2022

(51) Int. Cl. A61K 8/64; A61K 8/34; A61K 8/44; A61K 8/46; A61K 8/58; A61K 8/67; A61K 8/73; A61K 8/86; A61Q 5/02; A61Q 7/00

(52) U.S. Cl. CPC A61K 8/64; A61K 8/34; A61K 8/44; A61K 8/442; A61K 8/46; A61K 8/58; A61K 8/673; A61K 8/73; A61K 8/86; A61Q 5/02; A61Q 7/00

(57) ABSTRACT

The present invention provides a hair growth essence and its preparation method; including the following weight percentages of each component: water 89.24%, PEG-400 7%, dextran 1%, guanine 0.5%, MSM 1%, biotin tripeptide-1 0.01%, blue copper peptide 0.35%, panthenol 0.5%, zinc gluconate 0.1%, phenoxyethanol 0.15%, chlorphenesin 0.15%. The present invention also relates to the aforementioned preparation method of the hair growth essence. The components covered by the present invention are safe and non-toxic, the formulation is reasonable, containing effective ingredients such as PEG-400, citrulline, biotin tripeptide-1, blue copper peptide and zinc gluconate; it can effectively strengthen hair roots, regulate metabolism and promote new hair growth, while the main ingredient PEG-400 has a dispersive effect; blue copper peptide has the effect of improving skin repair ability, promoting skin absorption, deep skin wake-up and so on.

### TECHNICAL FIELD

The present invention relates to the technical field of hair growth essence; in particular, it relates to a hair growth essence and its preparation method.

### BACKGROUND

Shampoos generally use triethanolamine of fatty alcohol sulfate and mixed salt of hydroxylamine, lauramide mipa, formaldehyde, polyoxyethylene, lanolin, fragrance, color and water as raw materials. The basic function of shampoo is to clean, which simply can not solve the problem of pathological dandruff, hair loss, etc., many “functions” are only business marketing gimmicks. For the current shampoo on the market, the focus on the effect of hair softness can make it greasy, dirty and collapsed easily. The focus on the effect of hair refreshing can also make the hair rough, dry, fly apart with poor repair performance easily. The two mentioned above contradict each other and are difficult to achieve at the same time. Surfactants used in shampoo mainly include: sodium fatty alcohol polyoxyethylene ether sulfate AES (ammonium), coconut oil diethanolamide (6501), betaine (CAB35), quaternary amine softener, amino acid surfactant, SLS and some functional additives: silicone slip and shine agent, glyburide anti-dandruff and anti-zooing agent, pearlescent agent, preservative, flavor, color, etc. It is difficult for the existing shampoo to provide better care effect, therefore, there is a need to design a plant peptide shampoo.

### SUMMARY

The purpose of the present invention is to provide a hair growth essence and its preparation method.

The present invention is implemented by the following technical solutions:

The present invention relates to a hair growth essence, including the following weight percentages of each component:

| | |
|---|---|
| Water | 89.24%, |
| PEG-400 | 7%, |
| Dextran | 1%, |
| Citrulline | 0.5%, |
| MSM | 1%, |
| Biotin tripeptide-1 | 0.01%, |
| Blue copper peptide | 0.35%, |
| Panthenol | 0.5%, |
| Zinc gluconate | 0.1%, |
| Phenoxyethanol | 0.15%, |
| Chlorphenesin | 0.15%. |

The role of each of the components mentioned above in the present invention is as follows: PEG-400 covered by the present invention is polyethylene glycol, which is non-toxic, non-irritating, with goodwater solubility, and is well soluble in many organic components. The invention employs PEG-400, which will be fully compatible with other components and fully dissolved; in addition, the PEG-400 covered by the present invention has excellent lubricity. Moisturizing property, dispersivity; it is used in the components of the present invention to give full play to its excellent properties for adhesive, antistatic agent and softener, etc.

Dextran covered by the present invention has high specific rotation; partial hydrolysis mainly yields isomaltose. Provides a shiny feeling to the hair growth.

Guanine covered by the present invention is an effective substance with health care function, which can improve the immunity of human body; it also has the effect of treating high blood pressure and reducing body fatigue.

MSM covered by the present invention is dimethyl sulfone, an organic sulfide, which has the ability to enhance the production of insulin in human body while playing a role in promoting the metabolism of sugar.

Biotin tripeptide-1 covered by the present invention is a tripeptide combining vitamin H and Matrikine series GHK, which can stimulate the synthesis of collagen and laminin, and fix the hair in the dermal hair follicle to prevent hair loss.

Blue copper peptide covered by the present invention is blue copper peptide, which has the effect of improving skin repair ability, promoting skin absorption and deep skin wake-up, etc., specifically:
(1) Improve skin repair ability: blue copper peptide can promote the synthesis of skin matrix and enhance the regeneration ability of skin cell tissue, so that the skin can heal quickly when it is damaged.
(2) Promote skin absorption: the molecular structure of blue copper peptide is small, and most of them are small molecule proteins, which can be easily absorbed by skin, so the absorption and utilization of effective ingredients by skin can be accelerated by adding this substance in skin care products.
(3) Deep skin wake-up: when blue copper peptide penetrates into the bottom layer of skin, it is good for stimulating the vitality of cells, enhancing the activity of deep skin cells, making the skin soft and elastic, and regular use can help improve the luster and hydration of the skin.

Panthenol covered by the present invention can promote wound healing and anti-inflammation; the application of panthenol on hair can play a role in hair care, which can also prevent hair splitting as well as increase the density of hair and improve the luster of hair. Zinc gluconate covered by the present invention is usually able to treat patients with abnormal taste and metabolic disorders caused by zinc deficiency, and is also effective in the treatment of acne and malnutrition. This drug is mainly based on zinc gluconate, after taking this drug, it is beneficial for the absorption of nutrients in the body and the intelligence of children can also be improved. It can also accelerate growth and development and prevent the symptoms of stunting.

Phenoxyethanol covered by the present invention is a preservative with low skin sensitivity, it is added to cosmetics to act as an antibacterial and antiseptic agent and may also have a certain degree of emulsification effect that affects the viscosity of skin care products, which is a fairly safe preservative. It is employed as a substitute for formaldehyde, as formaldehyde is quite irritating, and after replacing it with phenoxyethanol, the irritation and damage to human body is significantly reduced. For some people with sensitive skin, the presence of a slight local burning and searing sensation may still occur. Sometimes it may be irritating to use the mucous membrane area of the eyes, and products containing phenoxyethanol should be avoided. However, in general, phenoxyethanol is still safe for the skin.

Chlorophenoxyethanol covered by the present invention can mainly play the role of disinfection and sterilization, and can inhibit the growth of many bacteria, so it can play the role of hair care. The addition of this component to the hair growth essence covered by the present invention can eliminate some symptoms of inflammation, such as a certain degree of relief for redness and itching to avoidance of unsafe use for allergic bodies.

The present invention also relates to the aforementioned preparation method of the hair growth essence, including the following steps:
S1, accurately weighing the component materials of the formulation ingredient in proportion to each other, while cleaning the production equipment;
S2, adding water to the stirring pot, increasing the temperature to 80-83° C., stirring evenly, holding for 20 minutes until completely dissolved, decreasing the temperature to 40-43° C. and cooling to room temperature.
S3, sampling and inspecting the prepared finished products mentioned above, and discharging and storing after passing the inspection.

The present invention has the following advantages:
(1) For the hair growth essence and its preparation method covered by the present invention, the components are safe and non-toxic, the formulation is reasonable, containing effective ingredients such as PEG-400, citrulline, biotin tripeptide-1, blue copper peptide and zinc gluconate; it can effectively strengthen hair roots, regulate metabolism and promote new hair growth, while the main ingredient PEG-400 has a dispersivity effect; blue copper peptide has the effect of improving skin repair ability, promoting skin absorption, deep skin wake-up and so on.
(2) All components of the present invention are natural environment-friendly, mild and have no side effects on human body, and can effectively improve hair loss prevention rate and hair growth promotion rate.

### DETAILED DESCRIPTION OF EMBODIMENTS

The following is a detailed description of the present invention in conjunction with specific examples. It should be noted that the following examples are only further descriptions of the present invention, but the scope of protection of the present invention is not limited to the following examples.

### Example 1

The present example relates to a hair growth essence, including the following weight percentages of each component:

| | |
|---|---|
| Water | 89.24%, |
| PEG-400 | 7%, |
| Dextran | 1%, |
| Citrulline | 0.5%, |
| MSM | 1%, |
| biotin tripeptide-1 | 0.01%, |
| blue copper peptide | 0.35%, |
| panthenol | 0.5%, |
| zinc gluconate | 0.1%, |
| phenoxyethanol | 0.15%, |
| chlorphenesin | 0.15%. |

The present invention also relates to the aforementioned preparation method of the hair growth essence, including the following steps:
S1, accurately weighing the component materials of the formulation ingredient in proportion to each other, while cleaning the production equipment;
S2, adding water to the stirring pot, increasing the temperature to 80-83° C., stirring evenly, holding for 20 minutes until completely dissolved, decreasing the temperature to 40-43° C. and cooling to room temperature.
S3, sampling and inspecting the prepared finished products mentioned above, and discharging and storing after passing the inspection.

The present invention has the following advantages:
(1) For the hair growth essence and its preparation method covered by the present invention, the components are safe and non-toxic, the formulation is reasonable, containing effective ingredients such as PEG-400, citrulline, biotin tripeptide-1, blue copper peptide and zinc gluconate; it can effectively strengthen hair roots, regulate metabolism and promote new hair growth, while the main ingredient PEG-400 has a dispersivity effect; blue copper peptide has the effect of improving skin repair ability, promoting skin absorption, deep skin wake-up and so on.
(2) All components of the present invention are natural environment-friendly, mild and have no side effects on human body, and can effectively improve hair loss prevention rate and hair growth promotion rate.

### Comparison Example 1

The preparation process is basically the same as that of example 1, the only difference is the absence of PEG-400, blue copper peptide, panthenol and zinc gluconate in the raw materials used to prepare the hair growth essence.

### Comparison Example 2

The preparation process is basically the same as that of example 1, the only difference is the absence of PEG-400, biotin tripeptide-1, panthenol and zinc gluconate in the raw materials used to prepare the hair growth essence.

### Comparison Example 3

The preparation process is basically the same as that of example 1, the only difference is the absence of PEG-400, biotin tripeptide-1 and blue copper peptide in the raw materials used to prepare the hair growth essence.

The effect of example 1 is compared with the effect of comparison example 1-3, see Table 1.

TABLE 1

| | | Comparison | Comparison | Comparison |
|---|---|---|---|---|
| | Example 1 | Example 1 | Example 2 | Example 3 |
| Hair Growth | 4.6 | 3.4 | 3.2 | 3.6 |
| Promotion Rate | | | | |
| Hair Loss | 4.7 | 3.2 | 3.1 | 3.2 |
| Prevention Rate | | | | |

As shown by the data of example 1 and comparison example 1-3, PEG-400, biotin tripeptide-1, blue copper peptide, panthenol and zinc gluconate act synergistically during the use of the hair growth essence, synergistically increasing hair loss prevention rate and hair growth promotion rate.

The specific examples of the present invention are described above. It should be understood that the present invention is not limited to the above specific embodiments, and the person skilled in the art may make various variations or modifications within the scope of the claims, which does not affect the essence of the present invention.

1. A hair growth essence, characterized in that, comprising the following weight percentages of each component:

| | |
|---|---|
| Water | 89.24%, |
| PEG-400 | 7%, |
| Dextran | 1%, |
| Citrulline | 0.5%, |
| MSM | 1%, |
| Biotin tripeptide-1 | 0.01%, |
| Blue copper peptide | 0.35%, |
| Panthenol | 0.5%, |
| Zinc gluconate | 0.1%, |
| Phenoxyethanol | 0.15%, |
| Chlorphenesin | 0.15%. |

2. A preparation method of the hair growth essence according to claim 1, characterized in that, comprising the following steps:
S1, accurately weighing the component materials of the formulation ingredient in proportion to each other, while cleaning the production equipment;
S2, adding water to the stirring pot, increasing the temperature to 80-83° C., stirring evenly, holding for 20 minutes until completely dissolved, decreasing the temperature to 40-43° C. and cooling to room temperature.
S3, sampling and inspecting the prepared finished products mentioned above, discharging and storing after passing the inspection.
